(19) United States
(12) Patent Application Publication
(10) Pub. No.: US 2018/0338869 A1
(43) Pub. Date: Nov. 29, 2018

(54) ABSORBENT PRODUCT COMPRISING FOAM MATERIAL

(71) Applicant: SCA Hygiene Products AB, Göteborg (SE)

(72) Inventors: Åsa JÖNEGREN, Göteborg (SE); Pia KALENTUN, Göteborg (SE); Susanna EDRUD, Göteborg (SE); Morgan HANSSON, Göteborg (SE); Susanne ANDERSSON, Göteborg (SE)

(73) Assignee: SCA Hygiene Products AB, Göteborg (SE)

(21) Appl. No.: 15/580,851

(22) PCT Filed: Jun. 10, 2015
(86) PCT No.: PCT/SE2015/050672

§ 371 Date: Dec. 8, 2017

(51) Int. Cl. A61F 13/472; A61F 13/475; A61F 13/537

(52) U.S. Cl. CPC A61F 13/472; A61F 2013/530649; A61F 13/537; A61F 13/475

(57) ABSTRACT

Absorbent product includes a liquid permeable topsheet, a liquid impermeable backsheet, and an absorbent core enclosed between the topsheet and the backsheet, wherein the absorbent core includes a liquid inlet foam material located closest to the liquid pervious topsheet and an absorbent fibrous layer located closest to the impervious backsheet. The liquid inlet foam material has in the transversal direction of the absorbent product two edge regions and one central region located between the edge regions. The central region has a plurality of openings formed from a plurality of slits extending in the longitudinal direction of the absorbent product and that openings have been formed from the slits by expanding the continuous liquid inlet foam material in the transversal direction of the product, and that each edge region of the liquid inlet foam material has a width that is at least 5.0 mm and is free from openings.

### CROSS-REFERENCE TO PRIOR APPLICATION

This application is a § 371 National Stage Application of PCT International Application No. PCT/SE2015/050672 filed Jun. 10, 2015, which is incorporated herein in its entirety.

### TECHNICAL FIELD

The present disclosure relates to an absorbent product including a liquid permeable topsheet, a liquid impermeable backsheet, and an absorbent core enclosed between the topsheet and the backsheet. The absorbent core includes a liquid inlet foam material located closest to the liquid pervious topsheet and an absorbent fibrous layer located closest to the impervious backsheet. The liquid inlet foam material has in the transversal direction of the absorbent product two edge regions and one central region located between the edge regions.

### BACKGROUND

For absorbent products such as sanitary napkins there are high requirements that they are discreet, soft and comfortable to wear and at the same time have a reliable security against leakage.

For sanitary napkins intended to absorb menstrual fluid being more viscous than urine, it is often more difficult for the body fluid to reach the absorbent core below the top sheet. Menstrual fluid may easily move around on the user facing side of the top sheet under the influence of gravity, motion and pressure by the user. Migration of menstrual fluid to the edges of the product increases the likelihood of leakage, and further smears the menstrual fluid against the skin of the user making cleanup more difficult.

It is also desirable that the products used for absorbing menstrual fluids are able to give the user a feeling of secureness and a visual impression that the menstrual liquid is absorbed by an absorbent core.

### DESCRIPTION

Absorbent product including a liquid permeable topsheet, a liquid impermeable backsheet, and an absorbent core enclosed between the topsheet and the backsheet. The absorbent core includes a liquid inlet foam material located closest to the liquid pervious topsheet and at least one absorbent fibrous layer located closest to the liquid impervious backsheet. The liquid inlet foam material has in the transversal direction of the absorbent product two edge regions and one central region located between the edge regions. The central region has a plurality of openings formed from slits extending in the longitudinal direction of the absorbent product and the openings have been formed from the slits by expanding the continuous liquid inlet foam material in the transversal direction of the product, and the edge region of the liquid inlet foam material has a width that is at least 5.0 mm. The edge regions are free from openings.

The continuous liquid inlet foam material sheet has been expanded in the direction transversal to the slits to obtain the openings from the slits. The liquid inlet foam material sheet may be extended to the same degree across its whole length.

The continuous liquid inlet foam material may be extended 30% to 200% from the original length of the liquid inlet foam material in its unexpanded state.

For a liquid inlet foam material having a slit length of 10.0-15.0 mm, the continuous liquid inlet foam material in the central region may be extended 50% to 190% of the original length in the central region of the liquid inlet foam material in its unexpanded state, and, in particular embodiments, extended 50% to 130%. For a liquid inlet foam material having a slit length about 5.0 mm, the continuous liquid inlet foam material may be extended 30% to 60% of the original length in the central region of the liquid inlet foam material in its unexpanded state. For a liquid inlet foam material having a slit length about 10.0 mm, the continuous liquid inlet foam material in the central region may be extended 50% to 130% of the original length of the unexpanded liquid inlet foam material in its central region. For a liquid inlet foam material having a slit length about 15.0 mm, the central region of the liquid inlet foam material may be extended 80% to 190% of the original length of the unexpanded liquid inlet foam material in the central region.

It has been shown that the pliability of the absorbent product is improved compared to an absorbent product having a foam material without openings. Another advantage with openings in the liquid inlet foam material is that the liquid inlet is improved.

The slits in the foam material extends in the longitudinal direction of the absorbent product, and the liquid inlet foam material is, in the transversal direction of the absorbent product, in an extended condition. The openings are formed by an expansion of the liquid inlet foam material in the transversal direction of the absorbent product. For absorbent products that are produced in the length direction it may be easier to produce the absorbent products by expanding the liquid inlet foam material in the transversal direction of the absorbent product instead of expanding the liquid inlet material in the longitudinal direction of the absorbent product. For absorbent products that are produced in the length direction, the step of expanding the liquid inlet foam material in the transversal direction of the absorbent product to form openings from the slits will be a step in the process of manufacturing the absorbent products that does not need, to the same extent as if the extension step is made in the longitudinal direction of the absorbent product, to be synchronized with other steps in the process.

An advantage by expanding the liquid inlet foam material in the transversal direction of the absorbent product, and also having edges without slits forming openings in the transversal direction, is that the liquid inlet foam material may be easier to grab and hold in the slit free edges during the expansion step.

The slit length may be between 5.0 to 20.0 mm.

The liquid inlet foam material may, according to one embodiment, be the top sheet or part thereof.

An advantage to have an absorbent product including a foam material is that such a product is experienced as soft and is also aesthetically pleasing for many users. The ability of a foam material to spring back, to return to substantially its original form after having been exposed to outer loading is also an important property. Since the foam material has a continuous structure, it exhibits good pliability and also ability to spring back following outer loading.

An alternative way of creating openings in a material would be to punch/perforate. However, a slitting method is advantageous over punching/perforating since no material is cut out from the web. This saves money due to less waste of material and also improves the handling in the production process by avoiding having a lot of small pieces cut out from the material that may otherwise contaminate both the process equipment and the final product.

An advantage with edges in the transversal direction of the liquid inlet foam material without openings is that a smooth surface against the user's skin along the edge regions in the longitudinal direction of the product is obtained. Another advantage is that the edges may function also as liquid edge barriers decreasing the risk for side leakage. Another advantage is that the liquid inlet foam material may be easier to grab and hold in the slit free edges during the expansion step.

The slit length may be 5.0-20.0 mm, 5.0-15.0 mm or 10.0-15.0 mm. For sanitary napkins intended to absorb menstrual fluid being more viscous than urine, it is often more difficult for the body fluid to reach the absorbent core than for absorbent products intended for urine. Menstrual fluid may easily move around on the user facing side of the top sheet under the influence of gravity, motion and pressure by the user. Migration of menstrual fluid to the edges of the product increases the likelihood of leakage, and further smears the menstrual fluid against the skin of the user making cleanup more difficult. By having a slit length that is at least 5 mm, or at least 10.0 mm the menstrual fluid will reach the absorbent core more easily.

The absorbent product has in its longitudinal direction two end parts and a crotch part enclosed between the end parts. The absorbent product may have a more narrow width in the crotch part or close to the crotch part than in the end parts. The reason for this is to shape the product to optimizing the fit to the body during use. The central region of the liquid inlet foam material may be located substantially parallel to a longitudinal center line in the longitudinal direction of the absorbent product, and not following the outer contour of the absorbent product. The width of the edge regions of the liquid inlet foam material in the absorbent product may therefore vary along the length of the absorbent product. However, the width of each edge region is at least 5 mm along the whole length of the absorbent product. If the edge region is less than 5 mm, even if only a short distance, it may be more difficult to attach the liquid inlet foam material to the other layers in the absorbent product. The narrowest width of the edge region may be between 5.0 and 15.0 mm.

The openings may have a longer dimension in the longitudinal direction of the absorbent product than in the transversal direction of the absorbent product. An advantage is that the oval shape of the slit openings in the longitudinal direction gives the user a visual impression of good liquid wicking in the longitudinal direction.

The width of the openings in the transversal direction of the absorbent product may be 0.3 to 1.0 of the length of the openings in the longitudinal direction of the absorbent product. The difference between the length of the slits and the length of the opening in the slit direction is that the length of the slits is measured on unexpanded material but the length of the openings in the longitudinal direction of the absorbent product is measured on the liquid inlet foam material in the absorbent product after the inlet foam material has been expanded.

The liquid inlet foam material may be hydrophobic. An advantage with a hydrophobic material is that hydrophobic edge regions will function as liquid barriers and will decrease the risk for edge leakage. The plurality of openings in the central region secure that the liquid reaches the absorbent layer(s) of the core below the liquid inlet foam material without the need of having a hydrophilic and absorbent foam material in itself. Also, in a particular embodiment, hydrophobic foam material close to the user's skin is from a skin care view since a hydrophobic and dry surface may decrease the risk for bacterial growth and skin irritations.

Foam material may have an open cell structure or a closed cell structure. Foam used as a liquid inlet layer in an absorbent product, is often foam with open cells so that the liquid easily enters the foam and also the absorbent core below. However, by having a plurality of openings, it is possible also to use foam including closed cells. An advantage with foam including closed cells is that the liquid will not so easily enter the foam structure itself, and therefore the foam material will be kept in a more dry condition than a foam material where the pores are connected with each other.

According to one embodiment is the average pore size of the liquid inlet foam material greater than the average pore size of the absorbent core layer below the foam resulting in a pore size gradient and a capillary suction force in the direction from the foam material towards the layer(s) of the absorbent core below the liquid inlet foam material.

The distance between the openings may be between 1.5 to 5.0 mm, or between 1.5 to 2.5 mm. An advantage with a rather short distance between the openings is that the inlet rate will improve.

The plurality of openings creates a pattern of the openings in the liquid inlet material. The slits may be provided in staggered rows extending in the longitudinal direction and having a slit distance B between the ends of two mutually sequential slits in the staggered row and a row distance C between two adjacent rows. The liquid inlet foam material may also have other slit patterns, and also more than one slit pattern. Different patterns of the openings may be formed by having slits with different length, or by having slits with different slit distance between the slits.

The total open area formed by the slit openings in the horizontal plane of the liquid inlet foam material in the central region may be 40-70% of the total area in the horizontal plane of the liquid inlet foam material in the central region.

The liquid inlet foam material may have substantially the same extension in the horizontal plane as the absorbent product. The central region with openings may extend along the whole length of the foam in the longitudinal direction of the absorbent product.

According to one embodiment is the absorbent product a sanitary napkin. The absorbent product may also be an incontinence shield, panty liner or a diaper. For example, to have a foam material for a premature baby diaper, may be advantageous since foam material may be very soft and flexible.

In order to reduce the problem that the absorbent product deforms during use, in particular in the crotch part of the product, it may be advantageous to increase the stiffness of the product. The absorbent core may include a stiffening element having a density greater than 100 kg/m³. Materials with a density greater than 100 kg/m³, so called stiffening elements, may be introduced into the product. Stiffening elements have the goal of retaining the shape of the product during use.

However, the skin in a user's crotch is relatively sensitive and a problem with stiffening elements is that they can scrape the user's skin, for example when the user moves. The foam's pliability and flexibility reduces the risk of scrapes. Liquid inlet layers of air laid, cellulose-based layers and liquid inlet layers of non-woven material do not have the same ability to reduce the negative effect of the stiff edges that a stiff cellulose-based absorption layer causes. Flexible foam materials may spring back and return to substantially their original shape after having been exposed to outer loading. Flexible foam materials also have a padding effect such that the foam material lines the stiff edges and creates a soft distancing element between the user's skin and the stiff edges of the absorbent fibrous layer.

A nonwoven and/or tissue layer may be located between the liquid inlet foam material and the absorbent fibrous layer. The nonwoven and/or tissue layer and the liquid inlet foam material may have different colors. The liquid inlet foam material may be colored or the nonwoven and/or tissue layer below the liquid inlet foam material may be colored. It may be an advantage to have different colors since the openings in the liquid inlet foam material will be visualized more clearly. Furthermore, if the absorbent core includes an absorbent layer having a shape with a less extension than the foam, a stiffening element may for example have a less extension than the foam, it is an advantage if a layer between the liquid inlet foam material and the stiffening element is colored since the outer contour of the stiffening element will not so easy be recognized by the user.

According to one embodiment is the absorbent product a sanitary napkin. The sanitary napkin may have a front part, a back part and a crotch part located between the back part and the front part, wherein at least one absorbent fibrous layer extends in the longitudinal direction of the product over the crotch part and at least a portion over the front part and that it has a width (M) in the transition between the crotch part and the front part which is less than 40 mm, and that the side edges of the liquid-absorbent, fibrous layer diverge in the direction from the transition between the crotch part and the front part to at least a portion over the front part.

The liquid inlet foam material may be laminated to a carrier material in its expanded condition so that the foam material is fixed to the carrier material with openings in its expanded condition. According to this embodiment, it may be an advantage if the foam or the carrier material is colored. The carrier layer may be a nonwoven and/or a tissue.

The carrier material may face the top sheet or be a part of the top sheet and the liquid inlet foam material may face the absorbent fibrous layer in the absorbent core. Alternatively, the carrier material may face the absorbent fibrous layer in the absorbent core and the liquid inlet foam material may face the top sheet or be a part of the top sheet.

An advantage having a colored nonwoven below the liquid inlet foam material is that the openings are visualized more clearly and will be more easily recognized by the user. If the absorbent fibrous layer has a less extension in the horizontal plan of the sanitary napkin than the liquid inlet foam material, it is advantageous to have a layer, for example a nonwoven, tissue or laminate thereof being colored since the less extension of the absorbent fibrous layer will not so easily be recognized by a user. If the absorbent core also is functioning as a shape element/stiffening element it may be an advantage to have a smaller extension on the stiffening element than the other layers of the sanitary napkin. By having a colored layer enclosed between the liquid inlet foam material and the stiffening element, the difference in size of the layers will not be recognized so easily by a user, which may give a more reliable impression of the product. A colored layer between the liquid inlet foam material and the absorbent fibrous layer may be liquid permeable and may be covering the whole surface of the absorbent product, or covering only a portion, for example covering only the crotch part, or only the central region of the liquid inlet foam material, either along the whole length of the central region of the liquid inlet foam material or only the crotch part of the central region of the liquid inlet material functioning as wetting zone.

### BRIEF DESCRIPTION OF FIGURES

FIG. 1A shows a top view of a liquid inlet foam material according to an embodiment of the invention before it has been expanded.

FIG. 1B shows a top view the liquid inlet foam material according to FIG. 1A after it has been expanded, i.e. after the slits have been opened to form openings.

FIG. 2 shows a cross-sectional view of a sanitary napkin according to an embodiment of the invention.

FIG. 3 shows the sanitary napkin of FIG. 2, seen from the side which will be facing the user when it is worn.

### DETAILED DESCRIPTION OF THE FIGURES

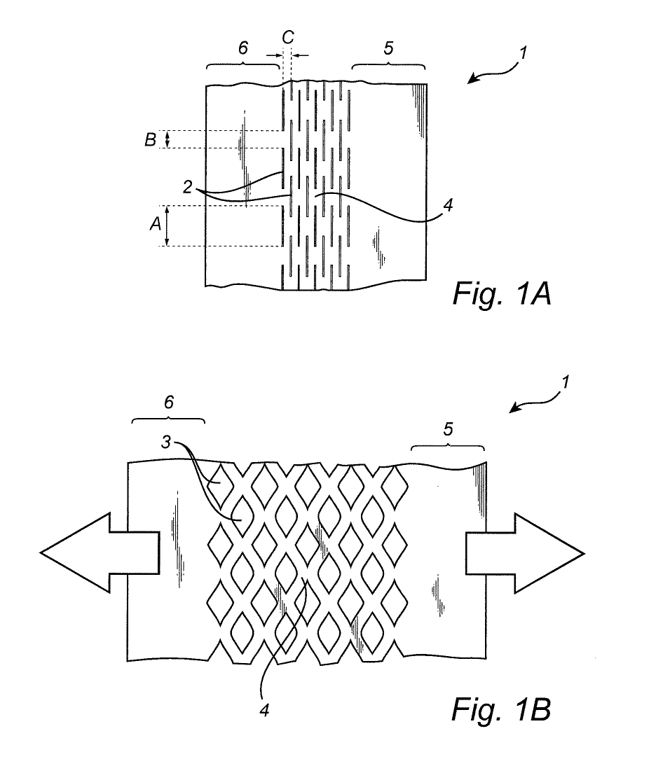

FIG. 1A shows a top view of a liquid inlet foam material according to an embodiment of the invention after it has been slitted but before it has been expanded. FIG. 1A shows a top view of the liquid inlet foam material 1 after provision of longitudinal slits 2. The slits 2 in FIG. 1A are straight, but may have any suitable shape such as for example wave-shaped. The slits 2 are provided in staggered rows extending in the longitudinal direction of the inlet material 1 having a slit length A of 5-20 mm. The liquid inlet material 1 has in its transversal direction a central region 4 with slits and two edge regions 5, 6 without slits.

FIG. 1B shows a top view of the liquid inlet foam material 1 according to FIG. 1A after it has been expanded in the direction transversal to the slit 2 direction, i.e. after the slits 2 have been opened to form openings 3.

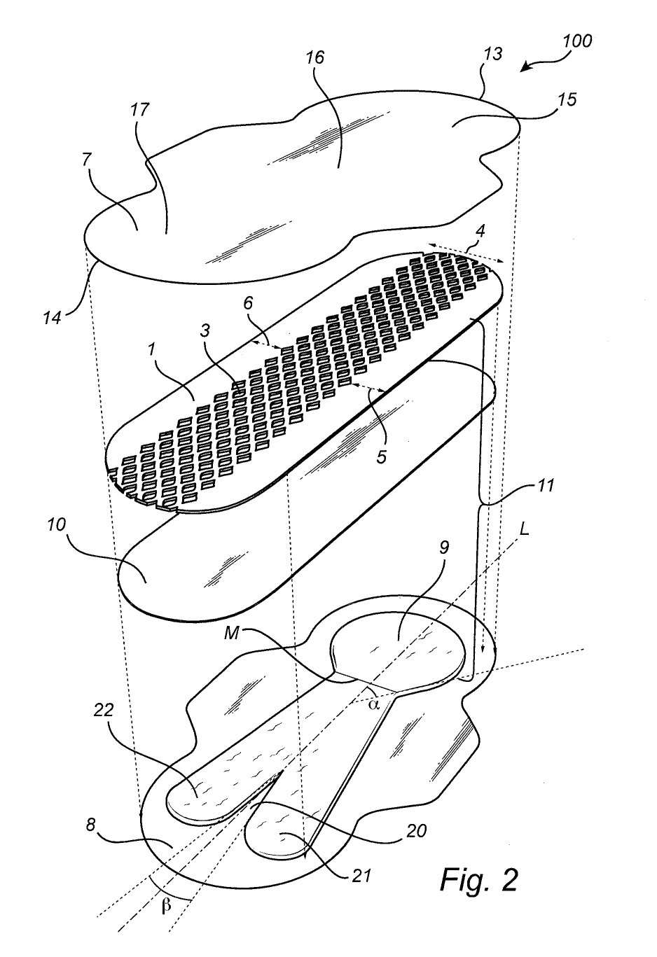

FIG. 2 shows a cross-sectional view of the different layers in the sanitary napkin according to an embodiment of the invention. The sanitary napkin 100 includes a liquid permeable topsheet 7, a liquid impermeable backsheet 8, and an absorbent core 11 enclosed between the topsheet 7 and the backsheet 8. The absorbent core 11 includes a liquid inlet foam material 1 arranged directly below the topsheet 7, an absorbent fibrous layer 9 directly above the backsheet 8, and a liquid pervious layer of nonwoven and/or tissue 10 enclosed between the liquid inlet foam material and the fibrous absorbent core. The sanitary napkin 100 has in its longitudinal direction a front end part 15, a back end part 17 and a crotch part 16 intermediate the front end part 15 and the back end part 17. The crotch part 16 is a portion which is intended to be placed against the crotch of a wearer during use of the product and to constitute the main acquisition area for body fluid that reaches the incontinence shield 100. The liquid inlet foam material 1 is located along the whole length of the sanitary napkin 100.

The topsheet 7 and the backsheet 8 of the sanitary napkin 100 extend together laterally outside of the absorbent core 11 along the whole circumference of the absorbent core 11. The topsheet 7 includes any material which is suitable for the purpose, i.e. soft and liquid pervious. Examples of commonly found topsheet 7 materials are nonwoven materials, perforated plastic films, plastic or textile mesh, and fluid permeable foam layers. Laminates including two or more topsheet materials are also commonly employed, as are top sheets including different materials within different parts of the fluid permeable wearer-facing surface.

The backsheet 8 is fluid impermeable. However, backsheet materials that are only fluid repellant may be used particularly in instances where relatively small amounts of urine are expected to be taken up. The backsheet 8 is commonly constituted by a thin, flexible, fluid-impermeable plastic film, but fluid-impermeable nonwoven materials, fluid impermeable foams and fluid impermeable laminates are also contemplated within the scope of the invention. The backsheet 8 may be breathable, implying that air and/or vapor may pass through the backsheet 8. Furthermore, the backsheet 8 may have an outer, garment-facing surface of a textile material such as nonwoven.

The absorbent fibrous layer 9 may be made up of any suitable absorbent or fluid uptake material as known in the art, such as one or more layers of cellulose fluff pulp, fibre waddings, etc. The absorbent fibrous layer 9 may also contain particles of highly absorbent polymer material, commonly known as superabsorbents, which are materials having the ability to absorb and retain large quantities of fluid upon formation of a hydrogel. The superabsorbents may be mixed with cellulose fluff pulp and/or may be arranged in pockets or layers in the absorbent fibrous layer 9. The fibres may be pulp fibres and the superabsorbent material may be polyacrylate-based particles.

The absorbent core 11 may further incorporate components for improving the properties of the absorbent core 11. Some examples of such components are binder fibers, fluid-dispersing materials, wetness indicators etc., as known in the art. The sanitary napkin 100 has an elongate, generally rectangular shape when fully extended in all directions. Any suitable shape may be used for the absorbent product, such as hourglass shape, trapezoidal shape, etc.

The sanitary napkin has a transversal rear end edge 14 intended to be orientated rearwards during use of the absorbent article, and a front end edge 13 intended to be facing forwards towards the abdomen of the wearer. The sanitary napkin 100 may further include fastening means for fastening of the absorbent product inside a supporting pant garment, such as a pair of underpants. The fastening means may be in the form of two longitudinally extending bands of pressure sensitive adhesive arranged on the garment-facing surface of the backsheet 8. The fastening means can be covered by a releasable protective layer, e.g. a siliconized paper, a nonwoven or any other releasable material as is known in the art. Before placing the absorbent product in the supporting pant garment, the protective layer is removed from the fastening means to expose the adhesive and make it available for fastening to the pant garment.

The fastening means is optional and may be omitted. When using an adhesive fastening means, any suitable adhesive pattern may be used such as full coating of the backsheet, one or more longitudinal adhesive band, transverse bands, dots, circles, curves, stars, etc.

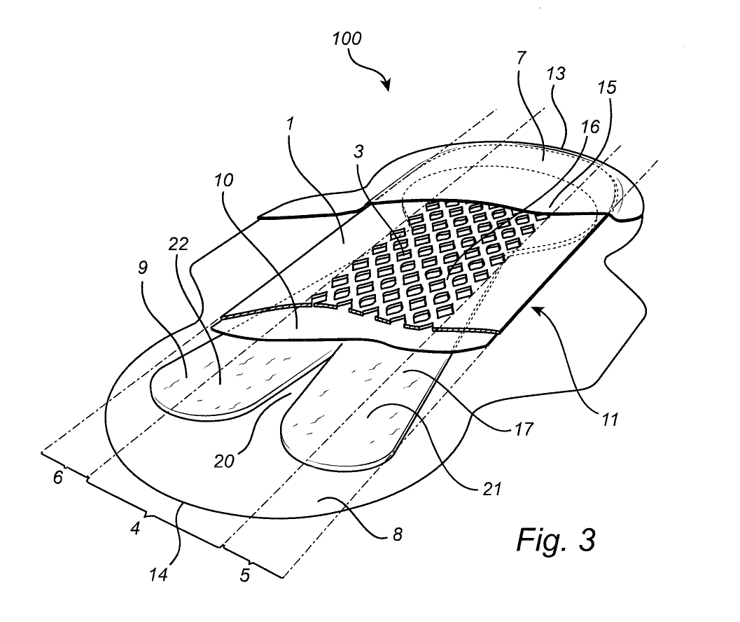

The inlet material 1 in FIG. 2 is situated above the nonwoven layer 10 and beneath and in direct contact with the topsheet 7. The absorbent fibrous layer 9 of the sanitary napkin 100 is situated between the nonwoven layer 10 and the backsheet 8. The absorbent core 11 includes the liquid inlet foam material 1, the nonwoven layer 10 and the absorbent fibrous layer 9, however, the nonwoven layer 10 is optional. The absorbent core 11 may also include more absorbent layers. The absorbent layers may have uniform thickness or may vary in thickness in different parts of the layers. Similarly, the basis weight and composition may vary within the absorbent layers. By way of example, the absorbent fibrous layer 9 may include a mixture of absorbent and/or non-absorbent fibers and superabsorbent material, wherein the ratio of superabsorbent material to fibers may vary in the layer. The absorbent core 9 may have any suitable shape, such as an hourglass shape with widened end portions and a narrow portion in the crotch portion, or a rectangular shape. The inlet material 1 in the sanitary napkin 100 illustrated in FIGS. 2-3 has a rectangular shape. The absorbent fibrous layer 9 includes a shape like a keyhole. The liquid inlet foam material 1 has a rectangular shape. The absorbent fibrous layer 9 is intended to function as a stiffening element and is designed to reduce the risk that the sanitary napkin 100 deforms in an uncontrolled manner.

The liquid absorbent layer has a width M in the transition between the crotch part 16 and the front part 14 which width is less than 40 mm, or 30-35 mm. Both side edges of the front part 15 diverge in a forward direction in the product from this transition M. In this manner it is prevented that the product is shifted backwards between the user's legs.

In FIG. 2, there is an angle between a line in the longitudinal direction of the product and each of the side edges of the absorbent fibrous layer in the front part 15 designated with α. In the case of a large angle α, for example, in the vicinity of 90°, the edges of the front part can scrape against the users groins and legs, thus creating discomfort for the user. The smaller the angle α is, the greater the risk that the product slides backwards in between the user's legs. At an angle below 30° this risk is unacceptably great. An angle between 35-45° gives the best balance between a firm hold and comfort.

FIG. 3 shows the sanitary napkin of FIG. 2, seen from the side which will be facing the user when it is worn.

The sanitary napkin 100 in FIGS. 2 and 3 is formed with a crotch length adapted to the user's anatomy. In a sanitary napkin in accordance with embodiments of the invention the fact was utilized that most women have a crotch length with a size of 80-100 mm. Along the crotch, where the user's body shape is substantially plane, the sanitary napkin is shaped so as to be relatively stiff laterally, i.e., it is sufficiently stiff so as to not be deformed laterally and form folds.

The width of the sanitary napkin in the crotch area is limited in front by the distance between the muscle sinews right in front of the user's groins. In the direction backwards from these transition areas to the end of the crotch part the width of the absorbent fibrous layer 9, that also functions as a stiffening element, can continuously increase to the size of 1.5 times the width M without the risk that the fibrous absorbent layer 9 scrapes the user in the crotch.

The absorbent fibrous layer 9 extends over the product's back part 17. The absorbent fibrous layer 9 has a recess 20 in the back part 17 and extending from its end edge and in the direction toward the crotch part 16 by means of which the product can fold along a longitudinally running line in the recess and by means of which the parts, the legs 21 and 22, that are located on both sides of the recess 20, become more flexible than the wider crotch part 16. This recess 20 is for obtaining a good adaptation to and pliability with the body. The fold which develops in the recess when the product is being used can pass into the user's posterior notch, thus offering a very good protection against leakage via the posterior notch, which type of leakage occurs when using sanitary napkins when the user is lying on her back. Furthermore, the recess 20 makes it possible that these legs 21, 22 can be mutually shifted vertically during various body movements, for example, when the user is walking.

In the exemplary embodiment shown in FIGS. 2 and 3 the recess 20 is wedge-shaped and symmetrically located relative to the longitudinally running line of symmetry L of the product and forms an angle β. This angle can vary within broad limits but is of course dependent on the shape of the back part 17.

The sanitary napkin 100 described in FIGS. 2-4 is only one illustration of an absorbent product suitable for the present invention.

### Examples

### Pliability Measurement

The force that is required to shape a product to match the body after being applied to underwear is measured when the product is folded/bent in a fixture designed for the purpose. The force is defined as the resistance that occurs when the product, which is resting on a Teflon-covered lower fixture, is folded into the fixture by downward-moving metal wires in an upper fixture. The product rests freely on two horizontal, parallel, Teflon-covered supports. The sample is pressed down at the centreline of the product between the two supports using a tensile tester. The maximum force is registered.

### Tested Products

The topsheet on the tested sanitary napkin has been a spunbond nonwoven with surface weight 22 g/m². The absorbent core on the tested sanitary napkin has been a pulp core mixed with polyacrylate superabsorbent particles where the surface weight of the pulp has been 255 g/m² and the surface weight of the superabsorbent has been 45 g/m². The backsheet has been a film with a surface weight 21 g/m².

Acquisition and distribution layer located between topsheet and absorbent core has been foam material according to following:

Foam

A: P30900 from Recticel without slits
B: P30900 from Recticel with slits
C: P32250 from Recticel without slits
D: P32250 from Recticel with slits

The slit length has been 10.0 mm. The slits have been provided in staggered rows extending in the longitudinal direction having a slit distance B between the ends of two mutually sequential slits in the staggered row and a row distance C between two adjacent rows.

### Results

| Sanitary napkin | Load (N) |
|---|---|
| A (P30900 no slits) | 4.7 |
| B (P30900 with slits) | 1.7 |
| C (P32250 no slits) | 3 |
| D (P32250 with slits) | 1.4 |

The result shows that the products with foam material with slits are more pliable.

### Liquid Inlet Time

The method evaluates the ability of sanitary napkins to absorb large quantities of liquids which come in contact with the product over a short period of time—known as splashes.

The inlet time is defined as that from the start of dosing 3 ml of artificial menstrual liquid until all the liquid is absorbed by the product.

The product is laid flat and exposed to dynamic pressure. The liquid is applied in one dose per product and the inlet time is registered by software.

The topsheet on the tested sanitary napkin has been a spunbond nonwoven with surface weight 22 g/m². The absorbent core on the tested sanitary napkin has been a pulp core mixed with polyacrylate superabsorbent particles where the surface weight of the pulp has been 255 g/m² and the surface weight of the superabsorbent has been 45 g/m². The backsheet has been a film with a surface weight 21 g/m².

The acquisition and distribution layer located between topsheet and absorbent core has been liquid inlet foam material according to following:

Foam

E: MCF003 from Recticel no openings
F: MCF003 from Recticel with openings
G: T25090 from Recticel no openings
H: T25090 from Recticel with openings
I: XD4200A from Caligen no openings
J: XD4200A from Caligen with openings

The slit length has been 10.0 mm. The slits have been provided in staggered rows extending in the longitudinal direction having a slit distance B between the ends of two mutually sequential slits in the staggered row and a row distance C between two adjacent rows. Openings have been formed from the slits by expanding the continuous liquid inlet foam material in the transversal direction of the product.

### Result Inlet Time

| Product | Inlet time (sec) |
|---|---|
| E (MCF003 no slits) | 11.1 |
| F (MCF003 slits) | 7 |
| G (T25090 no slits) | 20.3 |
| H (T25090 slits) | 10.7 |
| I (XD4200A no slits) | 32 |
| J (XD4200A slits) | 9.2 |

The result shows that the liquid inlet time is shorter with liquid inlet foam material with openings than liquid inlet foam material without openings.

### Density, Thickness and Contact Angles

Density, thickness and contact angles have been measured on the foam material itself, so without openings. The density and the thickness have been measured with an applied pressure on the foam of 0.5 kPa. The contact angle has been measured with a DAT-equipment after 1 second.

The result is as follows:

| | Supplier | | | | Contact |
|---|---|---|---|---|---|
| | code | Supplier | Density(kg/m3) | Thickness(mm) | angle |
| 1 | MCF003 | Recticel | 81 | 2.1 | 87 |
| 2 | T25090 | Recticel | 24 | 2.6 | 112 |
| 3 | XD4200A | Caligen | 28 | 3.0 | 102 |
| 4 | P30900 | Recticel | 38 | 3.2 | below 90 |
| 5 | P32250 | Recticel | 40 | 3.0 | below 90 |

1. An absorbent product comprising:
a liquid permeable topsheet;
a liquid impermeable backsheet; and
an absorbent core enclosed between the topsheet and the backsheet, wherein the absorbent core comprises:
a liquid inlet foam material located closest to the liquid pervious topsheet, and
an absorbent fibrous layer located closest to the impervious backsheet,
wherein the liquid inlet foam material in the transversal direction of the absorbent product has two edge regions and one central region located between the edge regions,
wherein the central region has a plurality of openings formed from a plurality of slits extending in the longitudinal direction of the absorbent product, the openings have been formed from the slits by expanding the continuous liquid inlet foam material in the transversal direction of the product, and each edge region of the liquid inlet foam material has a width that is at least 5.0 mm and is free from openings.

2. The absorbent product according to claim 1, wherein the continuous liquid inlet foam material sheet has been extended to the same degree across its whole length.

3. The absorbent product according to claim 1, wherein the liquid inlet foam material, has been extended 30% to 200% from the original length of the unexpanded liquid inlet foam material.

4. The absorbent product according to claim 1, wherein the slit length is 5.0-20.0.

5. The absorbent product according to claim 1, wherein the most narrow width of the edge region is between 5.0 and 15.0 mm.

6. The absorbent product according to claim 1, wherein the openings have a longer dimension in the longitudinal direction of the absorbent product than in the transversal direction of the absorbent product.

7. The absorbent product according to claim 1, wherein the width of the openings in the transversal direction of the absorbent product may be 0.3 to 1.0 of the length of the openings in the longitudinal direction of the absorbent product.

8. The absorbent product according to claim 1, wherein the foam material is hydrophobic.

9. The absorbent product according to claim 1, wherein the distance between the openings is 1.5 to 5.0 mm.

10. The absorbent product according to claim 1, wherein the total open area formed by the plurality of openings in the central area in the horizontal plane is 40% to 70% of the total area of the liquid inlet foam material in the horizontal plane in the central area.

11. The absorbent product according to claim 1, wherein the liquid inlet foam material has substantially the same extension in the horizontal plane as the absorbent product.

12. The absorbent product according to claim 1, wherein at least one layer in the absorbent fibrous core has a density greater than 100 kg/m³.

13. The absorbent product according to claim 1, wherein a nonwoven and/or tissue layer is located between the liquid inlet foam material and the absorbent fibrous layer.

14. The absorbent product according to claim 13 wherein the nonwoven and/or tissue layer and the liquid inlet foam material have different colors.

15. The absorbent product according to claim 1, wherein the product furthermore comprises a front part, a back part and a crotch part located between the back part and the front part, wherein at least one layer of the absorbent core extends in the longitudinal direction of the product over the crotch part and at least a portion over the front part and it has a width in the transition between the crotch part and the front part which is less than 40 mm, and the side edges of the liquid-absorbent, fibrous layer diverge in the direction from the transition between the crotch part and the front part to at least a portion over the front part.

16. The absorbent product according to claim 1, wherein the liquid inlet foam material is laminated to a carrier material in its expanded condition so that the liquid inlet foam material is fixed to the carrier material in its expanded condition.

17. The absorbent product according to claim 16, wherein the carrier material is the top sheet or part thereof, and the liquid inlet foam material is facing the absorbent fibrous layer.

18. The absorbent product according to claim 16, wherein the carrier material is facing the absorbent fibrous layer of the absorbent core and the liquid inlet foam material is facing the top sheet.
